(19) United States
(12) Patent Application Publication
(10) Pub. No.: US 2019/0125011 A1
(43) Pub. Date: May 2, 2019

(54) TEMPERATURE SENSITIVE SURGICAL FACE MASK FOR IDENTIFYING AT RISK PATIENTS AND REDUCING VIRAL INFECTION

(71) Applicant: Thomas Jefferson University, Philadelphia, PA (US)

(72) Inventors: John Eisenbrey, Philadelphia, PA (US); Annemarie Daecher, Shamong, NJ (US)

(73) Assignee: Thomas Jefferson University, Philadelphia, PA (US)

(21) Appl. No.: 16/092,725

(22) PCT Filed: Apr. 11, 2017
(86) PCT No.: PCT/US2017/027003

§ 371 Date: Oct. 10, 2018

Related U.S. Application Data
(60) Provisional application No. 62/321,590, filed on Apr. 12, 2016.

(51) Int. Cl. A41D 13/11; A41D 31/02; G01K 11/12

(52) U.S. Cl. CPC A41D 13/1115; A41D 31/02; A61B 5/01; A41D 2400/32; A41D 13/1161; G01K 11/12

(57) ABSTRACT

A disposable face mask that changes color as an indicator of fever to provide hospitals with a quick and inexpensive method for triaging infected patients while limiting exposure to others.

### PRIORITY CLAIM

This application is a National Phase under 35 U.S.C. 371 of International Application No. PCT/US2017/027003, filed Apr. 11, 2017, which claims the benefit of provisional application the benefit of U.S. Provisional Application Ser. No. 62/321,590 filed Apr. 12, 2016, the disclosure contents of which are hereby incorporated by reference in their entirety.

### FIELD OF INVENTION

The present invention is generally related to surgical masks and devices used for identifying and indicating an elevated temperature of a person wearing a mask.

### BACKGROUND OF INVENTION

Numerous viral infections exist in which transmission of the virus is active during the presentation of fever. Sterile face masks are one inexpensive and effective commercially available mechanism for reducing viral transmission by limiting transmission of airborne particles and water droplets from coughing and sneezing. In the event of a large disease pandemic, hospitals require a way to quickly identify infectious patients while still limiting exposure to non-infected patron and staff.

Particular versions of face masks are described in the literature, including U.S. Pat. No. 3,288,138—entitled “surgical mask”, and U.S. Pat. No. 3,049,121—entitled “oronasal mask.” These masks provide protection to both the patient, practitioners and those near sick patients by preventing the spread of germs from one patient to another.

At least some sterile masks have utilized a concept of color change. For example, U.S. Pat. No. 4,488,547—entitled “face mask,” describes a disposable surgical mask that comprises a color changing material covering a portion of the surface that changes with accumulation of bacteria so that it can be disposed of upon expiration of its useful lifespan.

Other masks, some disposable and others not disposable. For example, US20040163648—entitled “Bio-mask with integral sensors,” which is directed to a gas mask for use with monitoring patients comprising sensors on the mask including temperature sensor.

Further is US 20060254592—entitled “respiratory mask,” which describes a disposable mask that comprises inhalation and exhalation valves to filter air coming into the mask. However, these masks suffer from several issues, including being cumbersome, expensive, and are not sufficiently targeted to both protect from the passage of bacterial and viral loads between patient and others, as well as being able to quickly and efficiently identify the presence or absence of fever.

Applicant proposes an inexpensive face mask that will alter in color when the wearer has active fever. This can be used by the incorporation of temperature sensitive dyes, and/or chemical or liquid crystals thermometers currently used for disposable thermometers.

### SUMMARY OF INVENTION

The embodiments of the invention disclosed herein are related sanitary masks that further comprise one or more dyes, that are color changed in the presence of an active fever, i.e. a temperature over about 38° C.

A facial mask having impregnated therein a first thermochromatic material, suitable to change colors at a predetermined temperature. In certain embodiments, the first thermochromatic material changes color at about 32° C.

Certain embodiments comprise two thermochromatic materials, wherein the first and second thermochromatic material, have a different temperature change point, for example at temperatures of 32° and 33° C. A third thermochromatic material may be further impregnated into said facial mask, wherein the third thermochromatic material changes color at about 34° C. Preferably, with two or more thermochromatic materials, the materials are separated in a pattern along at least a portion of the thermochromatic mask.

In preferred embodiments, a facial mask comprises an inner layer and an outer layer, and a first and second thermochromatic material, wherein the first and second thermochromatic materials are on the inner layer of the facial mask and are arranged in alternating patterns on at least a portion of the inner layer. A third thermochromatic material may be included in further embodiments on the inner layer and certain embodiments may include the outer layer having at least a portion that is transparent.

A further embodiment is directed towards a facial mask comprising a perforated portion of the mask, extending through the at least one layer of a facial mask, wherein the perforation is of sufficient size to allow passage of a thermometer through the perforation and into the mouth of a patient wearing the facial mask.

A method for determining fever in a patient comprising placing on a said patient a facial mask comprising a thermochromatic material; wherein the thermochromatic material has a color change point corresponding to a temperature that indicates an elevated temperature in a patient; wherein upon a change in color of the temperature sensitive material, confirmation of an elevated temperature is determined.

A method for determining a viral infection in a group of presenting patients, comprising, providing each patient with a facial mask, comprising a thermochromatic material having a temperature inflection point at about 1° C. above normal temperature for a non-febrile patient; allowing for the patients to wear the mask for a predetermined amount of time; assessing the change or lack of change in a mask; determining that those patients having a color change to the thermochromatic material are exhibiting a fever; and sorting patients based on the presence or absence of a fever.

A thermochromatic material having a temperature transition point at about 32-33° C., and suitable for administration to a material for facial application; wherein the thermochromatic material can be impregnated into materials for disposable or non-disposable face masks in a medical setting.

Further embodiments are directed to a mask comprising two or more dyes, each directed to a different temperature, to indicate specific temperatures. A first temperature may identify the lack of a fever, and a second temperature may assist in identifying the severity of or grades of fever, which can aid in identifying patients having a temperature significantly above normal.

Masks can also be tailored to the specific or common temperature for known viral or transmittable infections, wherein a specific temperature is created with a transition point at or near the expected temperature for certain illnesses. Thus, an ordinary flu virus may be differentiated from a dengue fever, malaria, small pox, avian flu, swine flu, etc., based on the expected temperatures of the exhaled breath displayed for each virus. Certainly some bacterial infections also identify with fever and these can also be tailored as appropriate for the expected temperature. Further embodiments may also include a bacterial or viral indicator embedded into the facial mask.

Further embodiments are directed to a dye that can be sprayed onto existing sterile face masks to indicate temperature of the person wearing the mask. Still further embodiments are directed to a method of determining whether a person is febrile, without the need to use a thermometer or contacting the patient, by providing a color changing dye onto a sterile face mask, wherein the dye changes colors to indicate the presence of a fever upon a threshold temperature being met.

A further embodiment comprises a surgical style mask comprising a first layer and a second layer; said first layer intended to be adjacent to the face and said second layer being open to the air on one side and adjacent to the first layer on the opposing side; said first layer comprising a temperature sensitive matrix suitable to identify, via a color change, an exhalation temperature from a wearer. In certain embodiments, the second layer is an insulating layer that provides a greater operating temperature for the surgical style mask.

The invention, therefore, provides for a mechanism to identify febrile patients and to quarantine or treat such patients based on the increased risk of contamination. Rapid identification of patients presenting with fever from a larger population of patients wearing sterile face masks provides a new and inexpensive way to manage patient intake and to reduce the risk of patient-patient transmission of active viruses. Patients with active fever could be identified by the color of their face mask or respiratory gear, without direct or near direct healthcare provider contact. This device provides a quick, inexpensive way to separate febrile from non-febrile persons while wearing respiratory protective gear. In event of viral epidemic, implementation of these masks can quickly triage patients while also limiting respiratory exposure to other patients and staff may be useful in reducing transmission of the virus.

Embodiments may also be directed to methods of monitoring response of fever in patients in isolation or required to wear protective face masks to reduce disease transmission, comprising use of a temperature sensitive mast to identify fever or absence of a fever in a patient. For example, fever in active tuberculosis patients could be monitored without direct contact with the patient. Multiple dyes/sensors can be incorporated into the mask if multiple fever readings are required (i.e. fever vs. no fever).

The invention meets an unmet clinical need by replacing two individual technologies (face masks and thermometers), each individually requiring patient interaction, with a single device. Accordingly, the combination of the two items allows for quicker identification of patients with fever with reduced personnel contact and, provides a new and effective way to identify febrile patients.

### BRIEF DESCRIPTION OF THE DRAWINGS

FIG. 1 illustrates a patient wearing the temperature sensitive mask.

FIG. 2 depicts a temperature sensitive mask having a nasal and oral temperature patch.

FIGS. 3A and 3B depict a temperature sensitive mask having a patch of temperature indication, wherein 3B depicts a change in the indicating material.

FIGS. 4A and 4B depict a temperature sensitive mask coated with a temperature indicating material, wherein 4B depicts a change in the indicating material.

FIGS. 5A and 5B depict a temperature sensitive mask coated with two temperature indicating materials, wherein 5B depicts a change in at least one of the indicating materials.

FIGS. 6A and 6B depict a temperature sensitive mask coated with a coordinate plan of temperature indicating materials, wherein 6B provides an example of an indicated temperature.

FIG. 7 depicts a side profile of a temperature sensitive mask adjacent a patient;

wherein the mask comprises a first and a second layer.

FIG. 8 depicts an exploded view of a mask having an inner and outer layer.

FIG. 9 depicts a facial mask having a perforation in the mask for insertion of a thermometer.

### DETAILED DESCRIPTION OF THE EMBODIMENTS

As used herein, the term “about” means within 5% of a stated number. Thus, About 20° C., means 19°−21° C.

As used herein, the term “thermochromatic material” or “temperature sensitive material” refers to a material that changes colors due to a change in temperature. For example dyes, inks, minerals, liquid crystals, inorganic materials, paints, and combinations thereof, can be utilized, as is known to a person of ordinary skill in the art.

As used herein, the term “color changing temperature” means the temperature of which the thermochromatic material changes from one color to another.

Use of facial masks in medical facilities can significantly reduce the spread of infectious diseases. A particular benefit of facial masks is that they are an inexpensive consumable that can be properly disposed of after use, all while reducing spread of disease. Widespread use of facial masks is prevalent in certain countries around the world, but used less frequently in others.

The facial mask works by preventing the spread of droplets of saliva from the mouth onto other surfaces or into the air. By preventing the spread of these droplets to the air and surfaces, contamination can be reduced, thus reducing the chance of patient to patient, or patient to caregiver transmission. However, not all patients are ill or contagious. A frequent sign of illness or a patient that is likely to be contagious is one whom also has a fever, or elevated temperature.

When a patient currently enters a medical facility, most protocols require the patient to sign-in, wait in a waiting room, and spend time around other potentially ill patients. Only after a certain period of time is the patient even given an initial overview for temperature or other issues that would benefit both the patient and others near the patient. Only at that point might separation or a face mask be provided in many instances. Thus, current methods involve the separate application of temperature indication and of coverage of the mouth and nose of the patient.

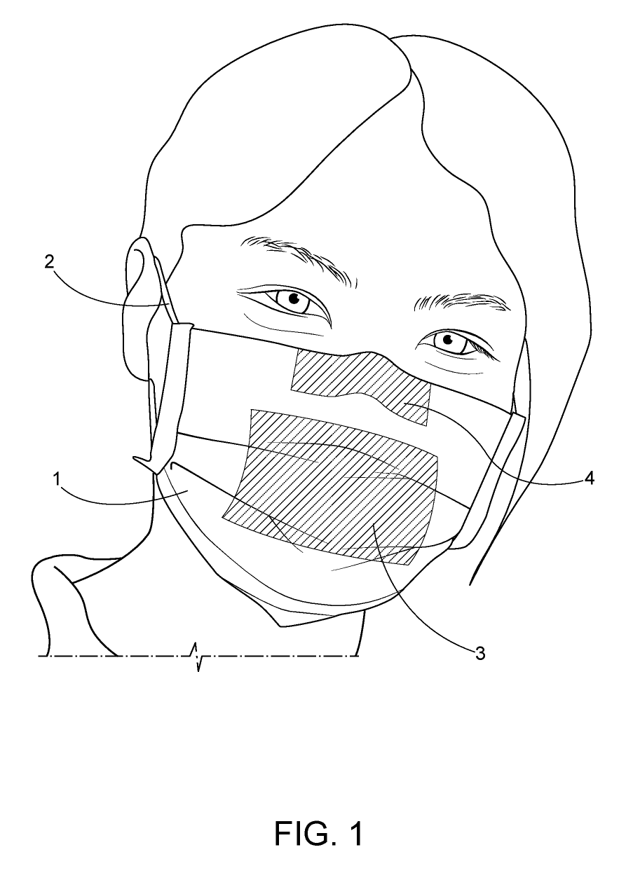

Currently, when a patient is given a mask, to take their temperature, removal of the mask is often required to place the thermometer in the patient's mouth. This process increases risk of contamination and spread of disease, which can be eliminated by the embodiments described herein. As disclosed in the embodiments therein, the device is a facial mask 1, as depicted in FIG. 1. The facial mask 1 can be placed over the mouth and/or nose of the patient, and secured with a securing device 2. For example, any one of the surgical masks, sterile masks, or sanitary masks are known to those of ordinary skill in the art. Known securing devices are tying strings, attachments around the ears, and elastic around the head. The facial mask 1 herein could immediately be given to patients as they walk into a healthcare setting. The facial mask 1 contains at least one coating panel, e.g. 3 or 4, corresponding to the mouth or the nose of the patient. These panels provide a color indicator that senses temperature from the mouth or nose and, will provide a change in color when a temperature above normal is indicated. Therefore, a patient with changes in their mask could then be escorted to treatment or quarantine facilities to avoid transmission to others, before the patient is seen by a medical practitioner.

In preferred embodiments, a face mask is a sterile or non-sterile facemask with a thermochromatic material, a dye, heat sensitive chemical or liquid crystal built in that changes the partial or entire color of the face mask when the wearer has active fever.

In manufacturing a face mask incorporating the thermochromatic materials, the face mask may be manufactured without the thermochromatic materials, and then a thermochromatic material can be sprayed directly on the inside or outside of commercially available face masks, which will change color when the wearer of the mask has active fever. Alternatively, during the manufacturing process, a layer of material to be incorporated into a temperature sensitive mask can be sprayed with, dipped into, or otherwise have the thermochromatic material applied to a layer of material that will become part of the face mask.

Therefore, a method of manufacture comprises manufacturing a facial mask; applying at least one thermochromatic material to said mask; drying the thermochromatic material. Alternatively, the method may include spraying on a thermochromatic material or dipping a layer of material of the facial mask into a thermochromatic material and then assembling the facial mask.

In certain embodiments, a thermochromatic material may be provided in a liquid or aerosol form, wherein the material can be applied onto a face mask. The thermochromatic material comprises a carrier, a thermochromatic color changing material suitable in said carrier, and is capable of being transformed from one color to another at a predetermined temperature. Therefore, a particular product may be a dye, paint, or other thermochromatic composition, having chemical changing properties, and that can be directly sprayed or coated on a commercially available face mask.

Preferred materials include leuco dyes, liquid crystal materials, or thermochromatic paints can be formulated in an appropriate carrier with a color change temperature suitable for the particular application. These materials can be purchased in certain commercial settings, or can be specifically manufactured for greater accuracy and a lower temperature range for the change in color. These examples were tested using a hair dryer to confirm color change. Specific concentrations of the dyes, paints, or thermochromatic pigments can be utilized with an appropriate carrier to create a material that will change at the necessary temperature point.

A preferred product, therefore, comprises a mask coated or impregnated with a thermochromatic material such as a paint or dye. The temperature change for the thermochromatic material is intended to identify an increased body temperature in the patient. The increased body temperature is appropriately measured from contact with the skin, i.e. an increase in skin temperature, and/or from exhalation from the nasal passages and/or the mouth. Accordingly, a mask sensing exhaled breath is appropriately coated or impregnated with a color changing material, at a bare minimum, around the nose and mouth sections of the mask. A mask sensing change in skin temperature is coated around the nose, chin, and cheeks. Certainly, the coating may be applied to the entire mask portion, or in individual spots.

Normal exhalation temperature in a non-febrile patient is approximately 32-36° C., with a mean temperature of about 35° C. However, the measured temperature, at even just a few cm away from the face and nose is considerably lower. Therefore, an increase of temperature should be measured at about 1° C. higher than the expected exhalation temperature for a non-febrile patient. Based on the tests performed, an appropriate temperature to identify an increase of about 1-2° C. was a color change threshold of 32° C. While this appears to be considerably below the expected temperature of 35° C./95° F., such a change is expected due to heat transfer with the surrounding environment. However, this ability to identify small (roughly 1° C.) changes is sufficient for identifying febrile patients and can be adjusted to specific temperature thresholds by varying specific dye combinations. It is preferable to have an overly sensitive mask, wherein temperature can be confirmed by a secondary device, than to omit a febrile patient.

Alternatively, the masks can be used to detect skin temperatures on the face, in addition to, or in combination with exhalation temperatures. Accordingly, the temperatures of the color changing material may be appropriately calibrated to measure an increase in skin temperature. Such masks should therefore fit snugly over the face, and temperature changing materials are suitably applied onto areas of the mask where skin contact is expected. For example, the upper cheeks, the nose, and the chin, all make up locations of expected skin contact with a mask. Facial skin temperature is highly dependent on environmental temperatures and time spent in that temperature environment. However a normal skin temperature is between about 32-35° C.

To confirm the absence or presence of fever in a patient, it may be necessary to include a further step of using a modern thermometer to test temperature in a more accurate manner. However, the ability to protect patients from spreading viral loads to other, non-symptomatic patients, and to further identify those patients who have highly elevated temperatures and may need immediate care is a major step in patient treatment. Temperature can be taken of a patient having a color change on their mask via oral, temporal, ear, or other suitable location using a standard thermometer.

In a preferred embodiment, the invention comprises a thermochromatic material, such as a dye, paint, or other color changing material, wherein the color changing point at between about 32° and 39° Celsius. The thermochromatic material can be applied to a material that will be suitably placed on or in close proximity to a skin surface of a patient, wherein a change in color of the material will signify the presence of an elevated temperature in the patient. Consequently, the device will prevent respiratory-based disease transmission, while also identifying febrile patients.

Further preferred embodiments comprise two or more thermochromatic materials being used at the same time, wherein a thermochromatic material changes at a temperature of only about 0.5° C. above the expected patient temperature, and a second thermochromatic material having a color changing property at about 1° C. or more above the expected patient temperature. Alternatively a first a 1° C. temperature change and a second a 2° C. temperature change above the expected temperature. Therefore, a two thermochromatic material system provides a wider range of temperatures to identify fever in a patient. Furthermore, in certain embodiments, the two temperature change can appropriately measure temperature in a patient to determine if the fever is not just elevated, but significantly elevated, as the second temperature dye will assist with not just identifying a patient with a fever, but identifying patients with highly elevated temperatures and in need of urgent care.

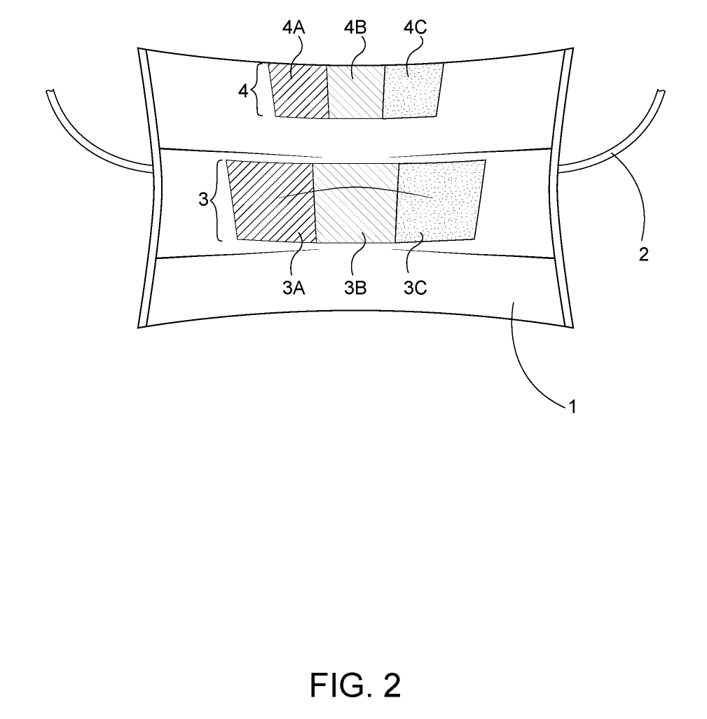

Three thermochromatic materials may also be utilized. For example, in FIG. 2, a facial mask 1 contains a mouth panel 3 and a nasal panel 4. Each of the mouth panel 3 and nasal panel 4 has three different sub-panels, each having a different temperature indication point, these are panels 3A, 3B, and 3C, and 4A, 4B, and 4C. The head strap 2, pictured assists to hold the facial mask 1 to the face of the patient. The combination of three different panels allows for different thermochromatic materials to be applied to the regions around the mouth and nose. For example, a first temperature may be at one degree C. above normal, a second at two degrees C. above normal, and a third at three or more degrees C. above normal. Thus, a mask may utilize a thermochromatic materials at, a temperature change of 32 C, 33 C, and 34 C, or the later at 35 C or higher.

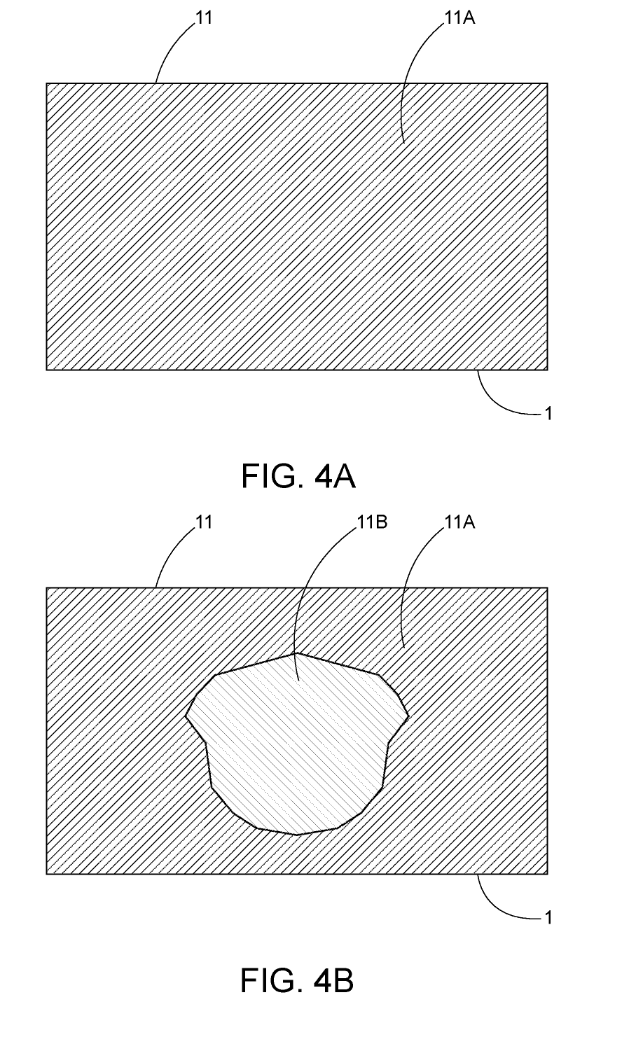

The panels 3A, 3B, and 3C, may be further aligned in strips, horizontally or vertically, so as to ensure representation among all points across the mouth, nose, and face. Or applied in a pattern along the mouth a nose portions, similar to FIG. 5. Alternatively, the materials combined together into a single panel, with the materials mixed together, yet able to independently change colors.

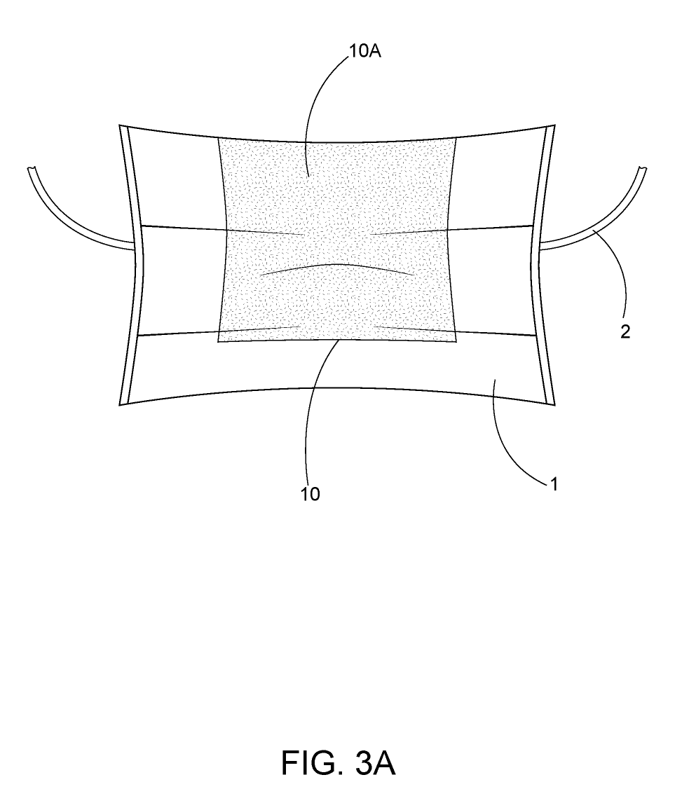

FIGS. 3A and 3B depicts an example of a facial mask 1 having a central panel 10 embedded with a thermochromatic material 10A. Upon application to the face, the thermochromatic material 10A will maintain its appearance if the wearer's core temperature is below 37.5° C. However, if the patient wearing the mask has an increased temperature, then a portion of the thermochromatic material will change to another color 10B, as depicted in FIG. 3B. The color change indicates a patient showing signs of an increased temperature, e.g. at 38° C. or higher.

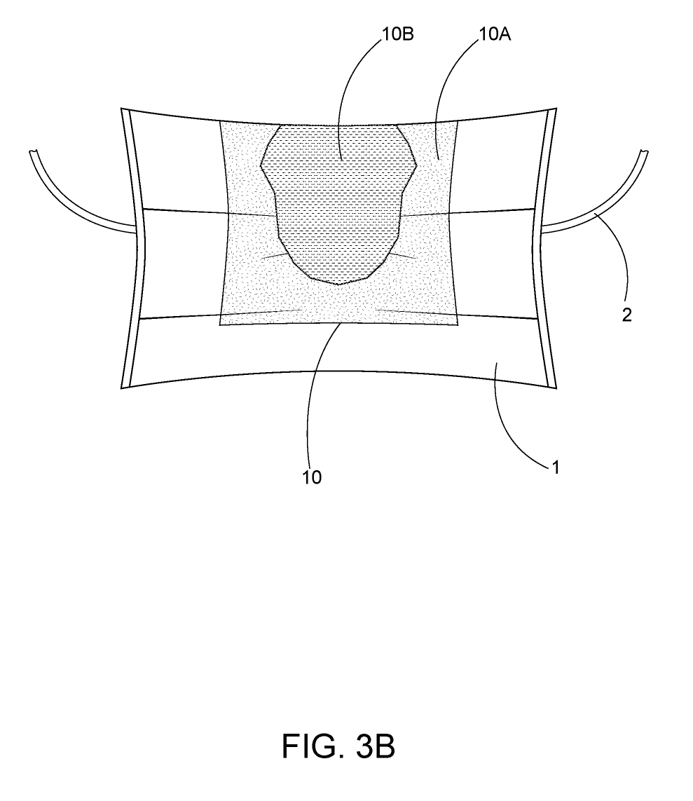

FIGS. 4A and 4B provide a variation of the embodiment of FIGS. 3A and 3B, wherein the face mask 1 has the entire panel 11 coated with a thermochromatic material 11A. Upon application to the face, a portion of the thermochromatic material 11A may change to a different color 11B upon contact with an increased temperature.

FIGS. 5A and 5B depict a further embodiment wherein a facial mask contains two thermochromatic materials 12 and 12A applied to the facial mask 1 in strip like patterns or alternating pattern designs. Upon application to an increased temperature, there is a color change 12B. Thermochromatic material 12 may change to one color, while thermochromatic material 12A may chance to a different color. Thus, the facial mask 1 of FIGS. 5A and 5B can be useful for detecting fever among a wide range of patients, for example, wherein the temperature of the first thermochromatic material 12 is at 32 C, and the second thermochromatic material 12A is at 33 C. Alternatively, the two color system can be utilized to differentiate between a low grade fever at a first temperature and a higher grade fever at a second temperature.

In generating the patterns on FIG. 5, or alternative patterns suitable for applying the thermochromatic materials to the surface of a facial mask, it is suitable to use stencils or other mechanisms to separately apply the first and second thermochromatic materials according to the predetermined pattern. Inclusion of a third thermochromatic material can be added and applied using the same stencils or mechanisms as is known to a person of ordinary skill in the art.

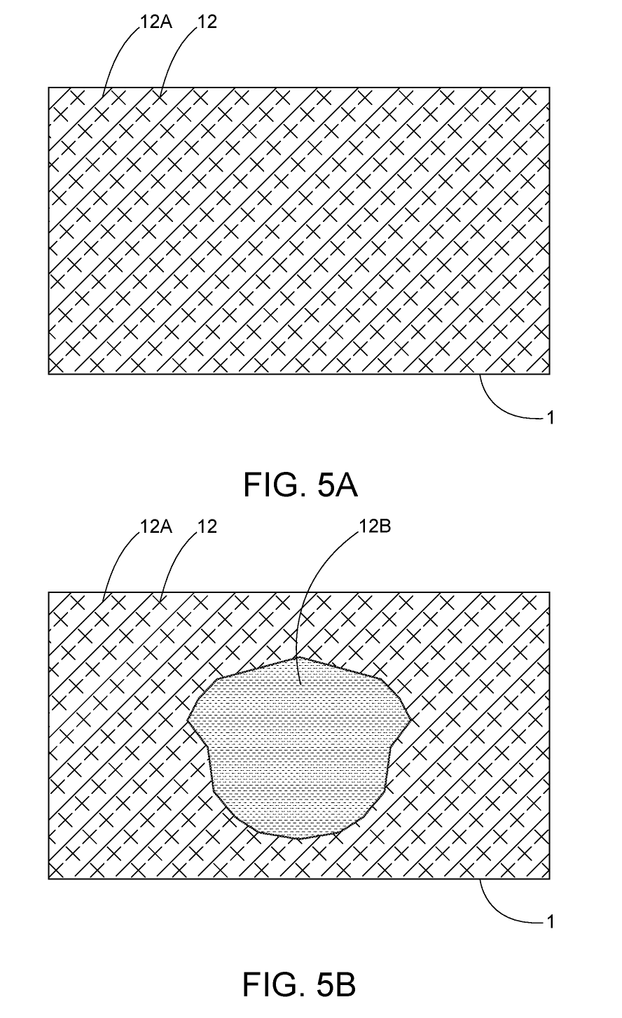

FIGS. 6A and 6B depict a further embodiment comprising a set of thermochromatic materials applied to a face mask 1. The color indicating materials, 9 and 15, are applied on a panel 13 wherein the vertical y axis 8, indicates partial temperature's, for example 0.1, 0.2, 0.3, 0.4 . . . 0.9. The numbers may be in increments of 0.1, 0.2, 0.3 or 0.5. Wherein the horizontal x axis 7 indicates whole temperatures, for example 31, 32, 33, 34, and 35 degrees Celsius (or corresponding Fahrenheit temperatures). Each of the circles can indicate a partial or whole temperature and wherein when a circle is illuminated, it includes a number within the circle that is visible. Thus, as identified in FIG. 6B, a temperature of a whole and partial degrees is indicated. This provides for a highly accurate level of temperature sensitivity. Such temperature readings are commercially available for direct application to a skin surface, and can be used on infants, for example to monitor temperature. Further temperature readings of this style are available as under the tongue thermometers as well.

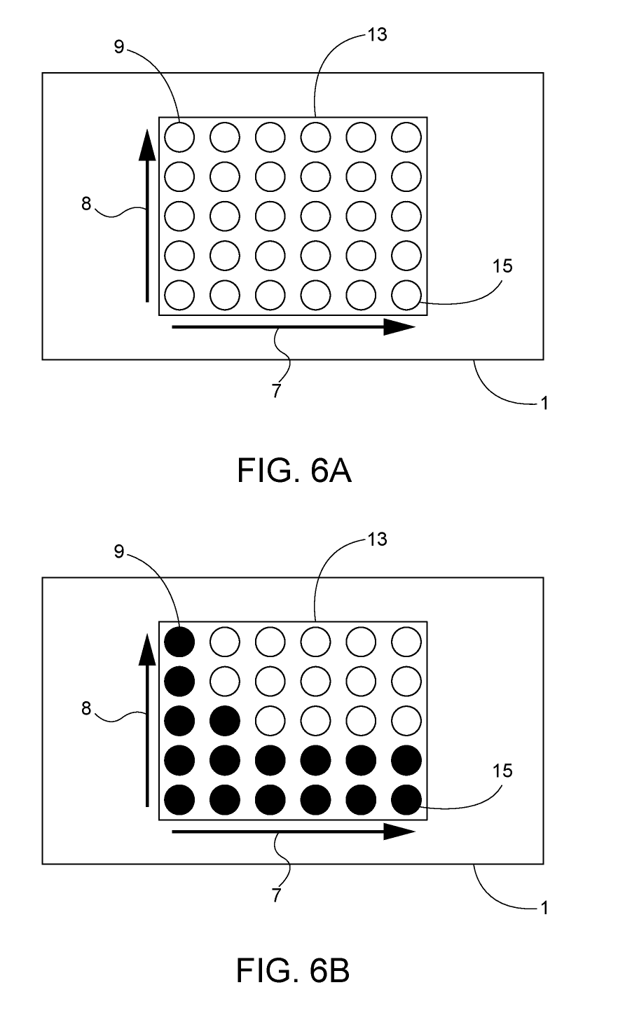

FIG. 7 depicts a further example of the construction of preferred embodiments of the facial mask 1. For example, a patient 30 applies a facial mask 1, to her face. The head strap 2 is applied to hold the facial mask 1 to the face. The mask comprises an inner layer 21 an outer layer 20 and a temperature sensing patch 27. The color sensitive material is positioned only on the interior of the mask so as to provide the greatest protection from spread of germs, but also to protect the temperature sensitive materials from ambient conditions. Accordingly, having an interior layer comprising the color sensitive/thermochromatic material may allow for use of the mask under temperature conditions that would otherwise prohibit the use of the mask.

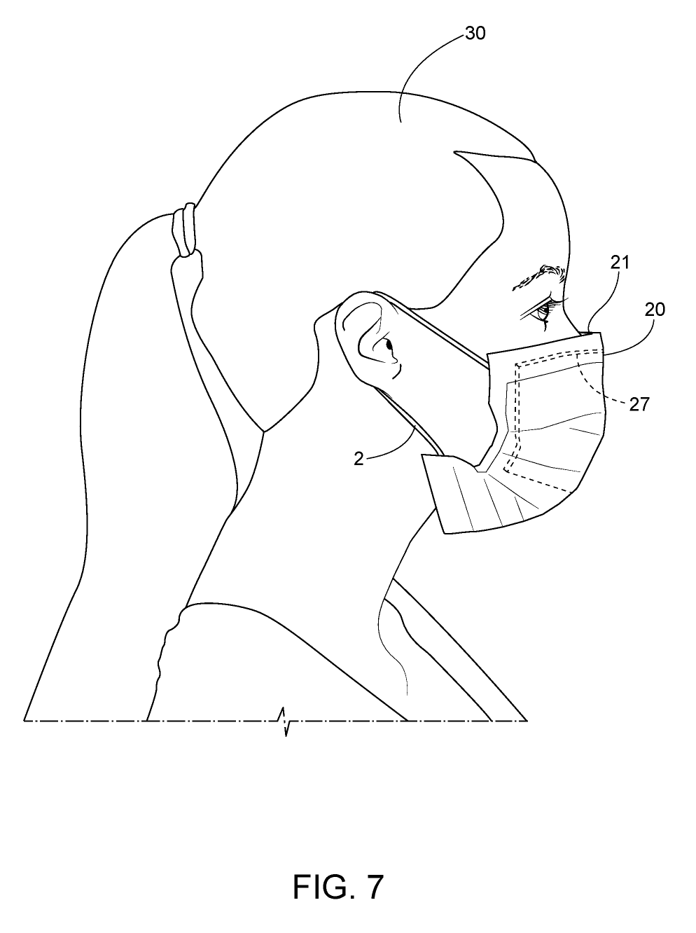

FIG. 8 further depicts the construction of the mask similar towards that of FIG. 7. An inner layer 21 comprises a temperature sensing patch 27. In FIG. 8 three different thermochromatic materials 22, 23, and 24 are embedded into the temperature sensing patch 27. For example, these can be arranged in strips, patches, regular patterns, or combined in a single material, allowing each to still change at a different temperature. The outer layer 20 comprises a normalizing material 25, suitable to prevent ambient temperatures from having significant impact on the temperature sensing patch 27. The normalizing material may be as simple as another layer of the facial mask, or may be a particular natural or synthetic material capable of both preventing spread of viruses or bacterial and also of thermal protection. The material, or certain portions may be transparent to allow for visual identification of the inner layer. The construction of the inner layer 21 and the outer layer 20 can be to simply affix them together, along the edge, or along any part of the faces. All or a portion of the normalizing material 25 may be transparent to allow for visual identification of the temperature sensing patch 27 of the inner layer 21.

Further embodiments are also directed to a thermochromatic material applied to a facial mask, and further comprising an imbedded disposable or reusable thermometer, positioned on the inside of the mask surface. Should a patient identify as febrile, the disposable thermometer may be utilized to property identify the actual oral temperature of the patient for admission to the hospital or medical location.

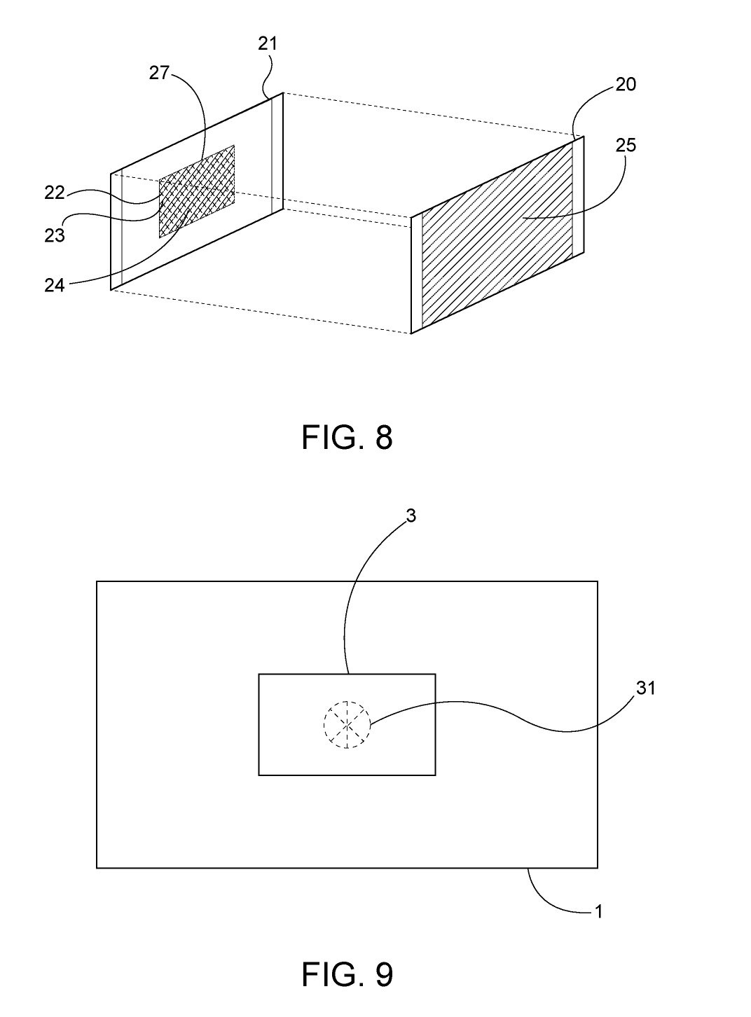

FIG. 9 depicts a further embodiment comprising a mask 1 having a temperature sensitive panel 3, and a perforated insertion point 31 at or around the mouth of the mask. This mask 1 thus provides that an oral thermometer can be inserted into the perforated insertion point 31 of the mask 1 for ease of a secondary temperature reading. Medical practitioners can also take temperature in other ways known to one of ordinary skill in the art, such as temporally or via the eardrum.

The perforated insertion point 31 may further comprise a rubber or plastic sealed opening, that is sealed from passage of vapors and breath from a patient, but can be penetrated by a standard thermometer for testing of oral temperature. The seal on the opening is such that after removal of the thermometer, the seal contains sufficient elasticity to re-seal the opening. This provides for a product having a combination of temperature sensing abilities, but also allowing for oral temperatures to be taken, without the need to undo the mask from the patient.

The mask is appropriately utilized in determining febrile patients from non-febrile patients within a medical setting; wherein a plurality of individuals are presented and each patient is given a color sensitive mask; wherein the color sensitive mask is impregnated or coated with a temperature sensitive dye, sufficient to identify whether the patient is expressing a temperature elevated above normal; wherein a change in the color of the color sensitive mask indicates an elevated temperature; and wherein said patients expressing an elevated temperature can be separated from the non-febrile patients in said medical setting.

The temperature sensitive mask may be utilized in a method of detecting an increased temperature in a patient, the method comprising instructing the patient to place the temperature sensitive mask over their mouth and nose; breathing into said mask, and detecting whether the mask indicates a change in color; wherein a change in color indicates an elevated temperature in the patient.

A method may also be utilized to determine fever in a group of patients comprising: providing masks having at least one thermochromatic material embedded into at least one layer of a temperature sensitive mask; instructing the patients to place the mask over their face and nose in the manner in which facial masks are ordinarily worn; allowing the patients to wear the masks for a predetermined period of time; after the duration of the predetermined time, reviewing the masks to determine whether any have had a color change; separating patients showing a color change on the masks so as to prevent the spread of disease between the patients.

The embodiments of the color sensitive mask, now being described can be modified accordingly to those of ordinary skill in the art to appropriately tailor the temperature and location of the temperature sensitive portions of the masks. Furthermore, the appropriate temperatures may vary based on the location of the mask to be utilized, including those having significant variance in ambient temperatures.

### EXAMPLES

Multiple prototypes of a facial mask were designed using five styles of disposable face masks and two temperature sensitive paints. Two blue thermochromatic paints were purchased, with designated color transition points (blue to white) of 32° C. and 33° C. All experiments were conducted in rooms at 20° C. Five grams of dye was added to 100 ml of white paint before being applied to each mask. After application and drying, the ability of all colored masks to turn from blue to white was confirmed by heating with a hair dryer. Dual-tie masks were then used for proof of concept studies. Skin temperature was monitored using a thermal camera. The first set of experiments consisted of the wearer sitting in a resting position for 20 minutes with images and temperatures obtained every 5 minutes. The second set of experiments involved the wearer running on a treadmill for 20 minutes with images and temperatures obtained every 5 minutes. Changes in body temperature and changes in mask color were then compared.

Several trials were conducted according to the materials and methods as described above. No change in skin surfaces were observed during resting trials. Masks of color change temperature of 32° C. and 33° C. were utilized to test these patients.

To test for temperature change, we asked participants to run on a treadmill, where running will increase both facial temperature and breathing temperature that is similar to the temperature profile of a patient exhibiting fever of about 38 or 39° C. Overall, a temperature of less than 2° C. temperature increase in the skin surface was observed over a 20 minute period of running (overall change in heart rate 90-100 bpm). This resulted in a change in the mask, for example as depicted in FIG. 3B.

Before the test started, the mask appeared as in FIG. 3A, wherein the thermochromatic panel 10 included unchanged thermochromatic material 10A. Neither mask changed color over a 20 minute period with the wearer sitting at rest, as shown in FIG. 3A. Accordingly, before the trial and while at rest, under normal exhalation, the mask does not change or indicate a thermochromatic change. Thus, a patient without elevated temperature will not show evidence of temperature change, and a patient with a fever will indicate a change in the thermochromatic material.

In further figures, the mask created with 33° C. showed little change in color over 20 minutes of treadmill running, with skin and exhalation at about 33-34 C. Accordingly, the small increase in the skin temperature was not clearly detected. However, a clear change of mask color was observed over the course of running with the mask created with 32° C. When comparing baseline to 20 minutes post running, large white areas within flaps, an overall fading of color, and pockets of completely white surface area around the nose and chin became apparent.

Applying a 32° C. sample to one point and a 33° C. to another point on a mask and applying a hair dryer to the mask shows that the 32° C. point changes faster than the 33° C., though bough eventually change. Obviously, the color changing temperature being lower, will change first, when hot air is applied. Though both will change when exposed to increased temperatures.

Thus, as we understand there is variability with all individuals on their specific internal temperature, masks incorporating a properly selected thermochromatic material can be utilized to detect fever in a patient. Furthermore, application of two or more thermochromatic materials together in a single facial mask provides for a wider range of temperatures so as to identify a change of about 1° C. to 2° C., for identification of fever in a patient.

What is claimed is:

1. A facial mask having impregnated therein a first thermochromatic material, suitable to change colors at a predetermined temperature.

2. The facial mask of claim 1, wherein the first thermochromatic material changes color at about 32° C.

3. The facial mask of claim 1, wherein the facial mask comprises a second thermochromatic material, having a different color changing temperature than the first thermochromatic material.

4. The facial mask of claim 1 comprising a second thermochromatic material impregnated into said facial mask, wherein the second thermochromatic material changes color at about 33° C.

5. The facial mask of claim 4, comprising a third thermochromatic material impregnated into said facial mask, wherein the third thermochromatic material changes color at about 34° C.

6. The facial mask of claim 1, wherein the first thermochromatic material is selected from a dye, ink, or paint.

7. The facial mask of claim 3, wherein the first and second thermochromatic materials are embedded in strips along at least a portion of the facial mask.

8. The facial mask of claim 3, wherein the first and second thermochromatic materials are embedded in an alternating pattern along at least a portion of the facial mask.

9. The facial mask of claim 3, wherein the facial mask comprises an inner layer and an outer layer, wherein the first and second thermochromatic materials are on the inner layer of the facial mask.

10. The facial mask of claim 9, wherein the first and second thermochromatic materials are arranged in alternating patterns on at least a portion of the inner layer.

11. The facial mask of claim 9, further comprising a third thermochromatic material on the inner layer, having a different color changing temperature from the first and second thermochromatic materials.

12. The facial mask of claim 11, wherein the first, second, and third thermochromatic materials are arranged in alternating patterns on at least a portion of the inner layer.

13. The facial mask of claim 9, wherein the outer layer has at least a portion that is transparent.

14. The facial mask of claim 9, further comprising a perforated portion of the mask, extending through both the inner and outer layers, wherein the perforation is of sufficient size to allow passage of a thermometer through the perforation and into the mouth of a patient wearing the facial mask.

15. A method for determining fever in a patient comprising placing on a said patient a facial mask comprising a thermochromatic material; wherein the thermochromatic material has a color change point corresponding to a temperature that indicates an elevated temperature in a patient; wherein upon a change in color of the temperature sensitive material, confirmation of an elevated temperature is determined.

16. The method of claim 14, wherein the temperature sensitive material is impregnated into the mask.

17. The method of claim 14, wherein the temperature sensitive material is coated into at least one layer of the mask.

18. A method for determining a viral infection in a group of presenting patients, comprising, providing each patient with a facial mask, comprising a thermochromatic material having a temperature inflection point at about 1° C. above normal temperature for a non-febrile patient; allowing for the patients to wear the mask for a predetermined amount of time; assessing the change or lack of change in a mask; determining that those patients having a color change to the thermochromatic material are exhibiting a fever; and sorting patients based on the presence or absence of a fever.

19. A thermochromatic material having a temperature transition point at about 32-33 C, and suitable for administration to a material for facial application; wherein the thermochromatic material can be impregnated into materials for disposable or non-disposable face masks in a medical setting.

20. The thermochromatic material of claim 18 which is selected from a dye, paint, or ink, liquid crystal, or mineral.
